(19) United States
(12) Patent Application Publication
(10) Pub. No.: US 2002/0119535 A1
(43) Pub. Date: Aug. 29, 2002

(54) Method for recombining polynucleotides

(76) Inventors: Steven C. Slater, Acton, MA (US); Wing-On Ng, Daly City, CA (US); Guo Hong Feng, Brookline, MA (US); Alex W. Monreal, Boston, MA (US)

Correspondence Address: Gail P. Wuellner, Monsanto Company - E2NA, 800 N. Lindbergh Blvd., St. Louis, MO, 63167, US

(21) Appl. No.: 09/746,432

(22) Filed: Dec. 21, 2000

(51) Int. Cl. C12P 19/34; C07H 21/04

(52) U.S. Cl. 435/91.2; 536/23.1

(57) ABSTRACT

Methods for recombining a polynucleotides of interest comprising the step of generating a recombinant polynucleotide by PCR amplification using as a template a pool of oligonucleotides having a chain terminating agent, e.g. ddNTP, at the 3′ end. Such oligonucleotides are preferably short, e.g. to provide for a higher degree of recombination. The PCR amplification is preferably carried out under conditions inducing mutagenesis in the amplified polynucleotide product. Recombined and mutagenized polynucleotides can be used to rapidly evolve cells and/or organisms to enhance or depress a phenotype of interest.

Disclosed herein are methods for recombining polynucleotides of interest including recombining mutagenized polynucleotides and methods of using such recombined polynucleotides, e.g. to accelerate genetic evolution of genes conferring a desired phenotype and/or encoding a polypeptide having an advantageous property.

### BACKGROUND OF THE INVENTION

Traditional molecular biological methods for generating novel genes and proteins generally involved rational or directed mutation. An example is the generation of a polynucleotide encoding a fusion or chimeric protein by using known restriction sites to combine functional domains from two characterized proteins. Another example is the introduction of a point mutation at a specific site in a polypeptide. Although useful, the power of these and similar methods is limited by the requirement for sequence or restriction map information to facilitate the mutagenesis, and by the limited number of variants that can be efficiently generated.

An alternative approach to the generation of variants uses random recombination techniques such as “DNA shuffling” as disclosed in U.S. Pat. Nos. 5,605,793; 5,811,238; 5,830,721; 5,837,458 and International Applications WO 98/31837, WO 99/65927, the entirety of all of which is incorporated herein by reference. In brief DNA shuffling entails cleaving a double-stranded polynucleotide template into random fragments which are denatured to provide a pool of single-stranded overlapping fragments which are used to reconstruct a template-sized polynucleotide, e.g. using polymerase. Typically the fragments are incubated with polymerase under conditions where the fragments form pairs with areas of identity sufficient for one member of a pair to prime replication of the other. The process is repeated in cycles of recombination and screening or selection to “evolve” individual genes, whole plasmids or viruses, multigene clusters, or whole genomes. Such techniques do not require the extensive analysis and computation required by conventional methods for engineering of polynucleotides and polypeptides. Moreover, DNA shuffling allows the recombination of large numbers of mutations in a minimum number of selection cycles, in contrast to traditional, pairwise recombination events. Thus, DNA shuffling techniques provide advantages in that they provide recombination between mutations in any or all of these, thereby providing a very fast way of exploring the manner in which different combinations of mutations can affect a desired result.

An alternative method of molecular evolution involves a staggered extension process (StEP) for in vitro mutagenesis and recombination of polynucleotide sequences, as disclosed in U.S. Pat. No. 5,965,408 and International Application WO 98/42832, the entirety of which is incorporated herein by reference. In the process a heterogeneous, single-stranded DNA population annealed with one or more random oligonucleotide primers is incubated with a polymerase for DNA synthesis. The DNA population is contacted with means for interrupting polynucleotide synthesis to produce partially double-stranded DNA fragments. The means for blocking or interrupting the amplification or synthesis process is by utilization of UV light, DNA adducts and DNA binding proteins. The partially double-stranded DNA is denatured to provide a mixture of single-stranded DNA sequences which are allowed to anneal. The annealed fragments are incubated with polymerase to form a pool of single-stranded fragments; and the denaturing, annealing and incubating cycle is repeated to form a pool of full length mutagenized polynucleotides.

An object of this invention is to provide an alternative and simpler method for recombining polynucleotides.

### SUMMARY OF THE INVENTION

This invention provides methods for recombining sequence in one or more polynucleotides of interest (e.g. genes or regulatory regions or both) by primer extension using as a template a pool of oligonucleotides having a chain terminating agent at the 3′ end, e.g. a dideoxynucleotide (ddNTP) or other compound whch is effective for blocking chain extension. The pool of 3′ blocked oligonucleotides can be produced from the polynucleotide of interest by

(a) copying the full length polynucleotide(s) of interest from a plurality of short random primers using ddNTP to terminate copy growth;

(b) cutting the full length polynucleotide(s) of interest with a series of restriction endonucleases into variable-length fragments and adding ddNTP at the 3′ end of each fragment; or

(c) copying the full length polynucleotide(s) of interest from one end primer into a pool of variable length oligonucleotides using ddNTP to terminate copy growth.

Preferably, such oligonucleotides are short, e.g. to provide for a higher degree of recombination. The terminating ddNTP can be included as a minor component of a deoxynucleotide (dNTP) mixture or can be added in one or more doses to terminate part or all of the synthesis. Enhanced genetic variation can be randomly induced by using a mixture of ddNTPs, e.g. a mixture comprising mostly ddGTP with minor amounts of ddATP, ddTTP and ddCTP or a different ddNTP mixture at similar concentrations. In preferred aspects of this invention, the synthesis of oligonucleotides is carried out under conditions inducing mutations in the oligonucleotide as compared to the template. Thus, the primer extension is preferably carried out under conditions inducing mutagenesis in the amplified polynucleotide product, e.g. using error prone polymerase or chain extension conditions.

One end primer for the original gene and polymerase are added to the pool of 3′ blocked oligonucleotides; the primer is extended by repeated cycles using the pool of templates. Extension on each primer terminates when the polymerase runs off the dideoxy block 3′ end of the template. After denaturing the progressively extended polynucleotide can switch templates for further partial extension until at least one full length polynucleotide is produced. Preferably, chain extension is carried out for a sufficient time to produce a plurality of polymorphic, full length, single-stranded recombinant polynucleotides. After a plurality of full length, single-stranded recombinant polynucleotides have been made, the other end primer(s) for original polynucleotide(s) of interest is used to make double-stranded polynucleotides, e.g. by PCR, for transfection and evaluation in vivo.

In another aspect of this invention, the template of a polynucleotide of interest can be a single polynucleotide or a plurality of polynucleotides having different nucleotide sequences, e.g. a family of homologous polynucleotides for the same or related species or different species. The plurality of homologous polynucleotides can be natural alleles or induced mutants.

### BRIEF DESCRIPTION OF THE DRAWINGS

FIG. 1 schematically illustrates an embodiment of recombination according to this invention.

FIG. 2 is a restriction enzyme map for preparing fragments of a gene to be recombined according to an embodiment of this invention.

FIG. 3 illustrates primers location and amplicon size for one embodiment of recombination.

### DETAILED DESCRIPTION OF PREFERRED EMBODIMENTS

As used herein the term “ddNTP” means a dideoxynucleoside-5′-triphosphate compound, e.g. dideoxyadenosine-5′-triphosphate (ddATP), dideoxycytidine-5′-triphosphate (ddCTP), dideoxyguanosine-5′-triphosphate (ddGTP), dideoxythymidine-5′-triphosphate (ddTTP) or dideoxyuridine-5′-triphosphate (ddUTP).

As used herein the term “dNTP” means a deoxynucleoside triphosphate compound, e.g. deoxyadenosine-5′-triphosphate (dATP), deoxycytidine-5′-triphosphate (dCTP), deoxyguanosine-5′-triphosphate (dGTP), deoxythymidine-5′-triphosphate (dTTP) or deoxyuridine-5′-triphosphate (dUTP).

As used herein the term “polynucleotide” means a large nucleic acid molecule, e.g. of DNA or RNA, typically greater than 1 kb, and includes genes, mRNA, cDNA or fragments thereof, e.g. exons or open reading frames of a gene of interest.

As used herein the term “oligonucleotide” means a short nucleic acid molecule of DNA, smaller than the corresponding polynucleotide, and having a chain terminating agent, e.g. a ddNTP, at the 3′ end of the molecule. An oligonucleotide will typically comprise greater than 5 nucleotides, e.g. deoxyribonucleotides The exact size will depend on many factors including the size of the corresponding polynucleotide of interest and the degree of nucleotide sequence shuffling desired in the recombined polynucleotide. For instance, for a given polynucleotide of interest there will be a plurality of oligonucleotides, e.g. greater than 5, more preferably at least about 10 or more, e.g. at least 20 and up to 100 or more oligonucleotides comprising fragments of a given polynucleotide of interest. For a given polynucleotide of interest there is preferably some degree of overlapping across the entire sequence of the polynucleotide. Preferably a higher number of oligonucleotides will provide a greater opportunity for nucleotide sequence shuffling in the polynucleotide of interest.

As used herein the term “primer” means a short nucleic acid molecule which is capable of acting as a point of initiation of synthesis when placed under conditions in which synthesis of a primer extension product which is complementary to a nucleic acid strand is induced, e.g. in the presence of nucleotides and an agent for polymerization such as DNA polymerase and a suitable temperature and pH. Useful DNA primers can be of random sequence or defined sequence. Although primers can have a variable length, especially useful primers will have a length in the range of 4 to 50 bases, e.g. specific primers often about 15 bases or longer or random primers often about 15 bases or shorter. The opportunity for incorporating polymorphism into the recombinant full length polynucleotide can be enhanced by synthesizing primers using a mixture of dNTPs at each step of the synthesis, i.e. a major amount of a single dNTP species and a minor amount of a mixture of other dNTPs, for instance a major amount of dATP and a minor amount of a mixture of dGTP, dCTP and dTTP.

In a preferred embodiment primers can be blocked at the 5′ end. Such blocking can be useful in preserving primers and the extended oligonucleotide when it is desirable to remove the original template, e.g. by digesting with a 5′ specific exonuclease prior to PCR amplification. Alternatively, in the case of RNA polynucleotide template and synthesized DNA oligonucleotides, the template can be removed by digesting with an RNase prior to PCR amplification. Alternatively, in the case of a DNA polynucleotide template and synthesized RNA oligonucleotides, the template can be removed by digesting with a DNase prior to PCR amplification.

In one aspect of the invention a pool of 3′ blocked oligonucleotides can be prepared by copying the gene from short random primers using ddNTP to terminate copy growth, e.g. in a moderated, enzyme-catalyzed, oligonucleotide polymerization synthesis. Short random primers, e.g. in the range of 6 to 15 nucleotides, can be a random set or a set designed for repetitive sequence in the polynucleotide(s) of interest. A sufficient number of the primers should hybridize to the template polynucleotide(s) of interest to permit synthesis of a pool of oligonucleotides from a mixture of dNTPs, wherein said polymerization is quenched at a selected time by adding ddNTPs to a polymerization synthesis mixture to form a pool of oligonucleotides having a ddNTP at the 3′ end. More particularly, a pool of 3′ blocked oligonucleotides can be prepared from random primers according to the following detailed procedure.

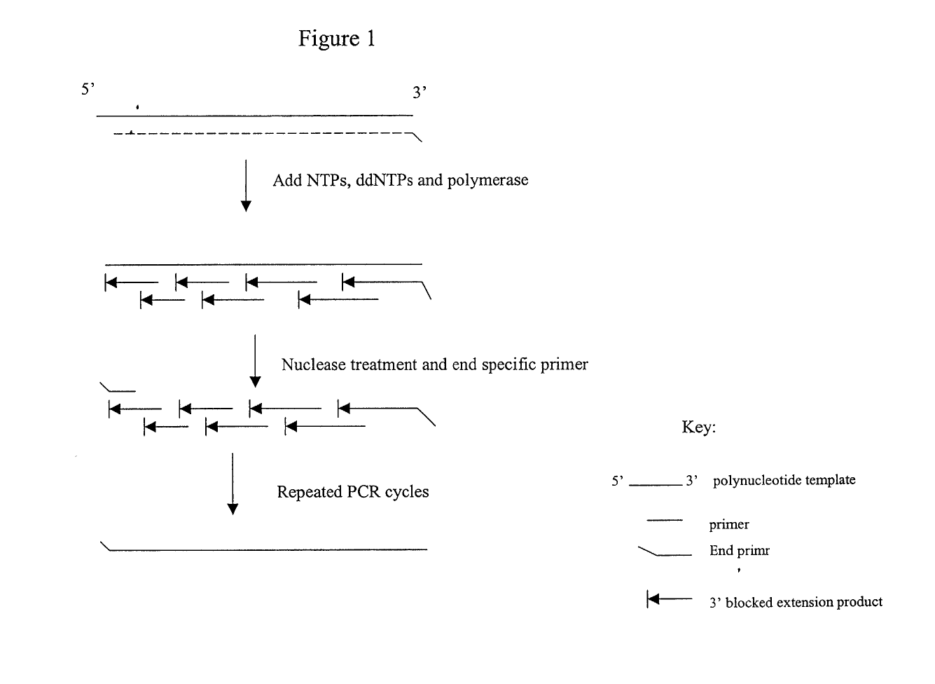

With reference to FIG. 1 there is schematically represented one embodiment of the method of this invention. A polynucleotide template is contacted with a plurality of primers, e.g. random sequence oligonucleotide primers in the range of 4 to 15 bases, and an end primer, e.g. a specific sequence oligonucleotide complementary to the sequence at the 3′ end of the template and preferably having a tail of sequence which does not match the template but which is used in subsequent amplification kits which are designed to initiate amplification with the sequence of the tail. In step A the primers are extended in the presence of dNTPs and ddNTPs, the ratios of which are selected to provide primers of a desired average length, determined by addition of a ddNTP blocking further extension at the 3′ end of the oligonucleotide. In many cases the extension is preferably conducted under conditions inducing mutations in the oligonucleotide.

In step B nuclease is added to remove the polynucleotide template, e.g. RNase to remove an RNA polynucleotide template. In the case of a DNA polynucleotide template, the primers can be designed with a 5′ block, then a 5′ specific DNA exonuclease can be used to remove the DNA polynucleotide template. Finally, in Step B primer specific for the ends of the polynucleotide template is added forming a pool of oligonucleotide and end specific primer.

In step C the oligonucleotides are extended by repeated cycles of primer extension using the pool of 3′ blocked oligonucleotides as templates. In many cases the primer extension is preferably conducted using polymerase and/or conditions that induce mutations. Repeated cycles of denaturation and annealing allow the extension products to switch between templates resulting in a recombined polynucleotide having at least one nucleotide which is different from the corresponding nucleotide in the original template polynucleotide of interest.

In another preferred aspect of the invention a pool of 3′ blocked oligonucleotides can be prepared from restriction endonuclease fragments of the template by adding a chain terminating agent to the 3′ end of the fragments. Preferably, the pool of oligonucleotides is prepared by combining groups of fragments prepared from different restriction endonucleases, e.g. at least two, more preferably at least four or more, restriction endonucleases. Restriction endonuclease digestion should be designed to produce overlapping fragments with recessive 3′ sticky ends, i.e. with a 5′ overhang which serves as a template for 3′ end blocking by addition of a ddNTP. For instance, aliquots of templates each of which has been cut by a distinct restriction endonuclease can be combined into a pool of overlapping fragments of the template. The pool of overlapping fragments can be blocked by adding ddNTP and a polymerase that does not have 3′→5′ exonuclease activity, e.g. Klenow Fragment (3′→5′ exo^{—}) polymerase, to add a blocking ddNTP to the recessive 3′ end of each fragment.

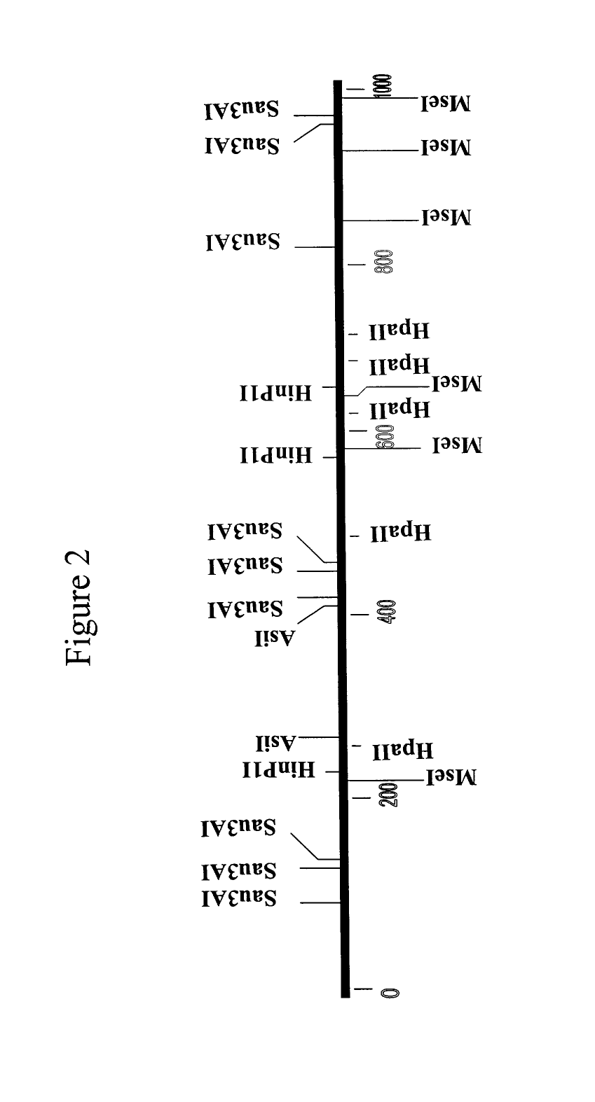

More particularly, a pool of 3′ blocked oligonucleotides can be prepared from restriction endonuclease fragments by the following detailed procedure and with reference to FIG. 2. In separate tubes, DNA (preferably, randomly mutagenized PCR amplified DNA) is digested with several separate restriction endonucleases, each leaving a 5′ overhang. DNA fragments are combined into a pool of overlapping DNA fragments and blocked by incorporating a ddNTP at the recessive 3′ end of the fragment and a mixture of one or more ddNTPs under reaction conditions appropriate for the polymerase. The 3′-end blocked fragments are purified away from the nucleotides.

Preferred restriction endonucleases are those that recognize tetra nucleotide sequences and can cut the tetra nucleotide sequence so as to leave a 3′ recessive sticky end. Such restriction enodonucleases are identified in Table 1.

TABLE 1

| Tetra nucleotide | Restriction endonuclease |
|---|---|
| ▾AATT | Tsp509 I |
| A▾CGT | Mae II |
| C▾CGG | Msp I; Hpa II |
| C▾TAG | Bfa I |
| ▾GATC | Dpn II; Mbo I; Sau3A I |
| G▾CGC | HinP I |
| G▾TAC | Csp6 I |
| T▾CGA | Taq I |
| T▾TAA | Mse I |

The arrrowhead ▾ indicates the point of cleavage.

The selection of restriction endonucleases is preferably based on analysis of the nucleic acid sequence of the gene of interest. It is preferable to cut the gene into fragments of variable size, e.g. in the range of 20 to 500 nucleotide base pairs, more preferably in the range of 40 to 400 base pairs, even more preferably in the range of 50 to 300 base pairs. In some cases it is useful to use multiple endonucleases for preparing digests of the gene of interest. In cases where a digest pool contains contains fragments outside of the desired length range, e.g. a plurality of short and/or long fragments, it is useful to separate the fragments, e.g. on a gel, to isolate the desired fragments. Digests can be combined to provide a pool of overlapping fragments with sufficient overlap of nucleotides to permit annealing of fragments necessary for template switching in chain extension reactions. In those cases where a point of cleavage is not adequately overlapped by another fragment, it is useful to design a synthetic oligonucleotide fragment based on the sequence of the gene of interest bridging the point of cleavage. The use of bridging oligonucleotides provides an opportunity for inserting a deliberate mutation into the sequence.

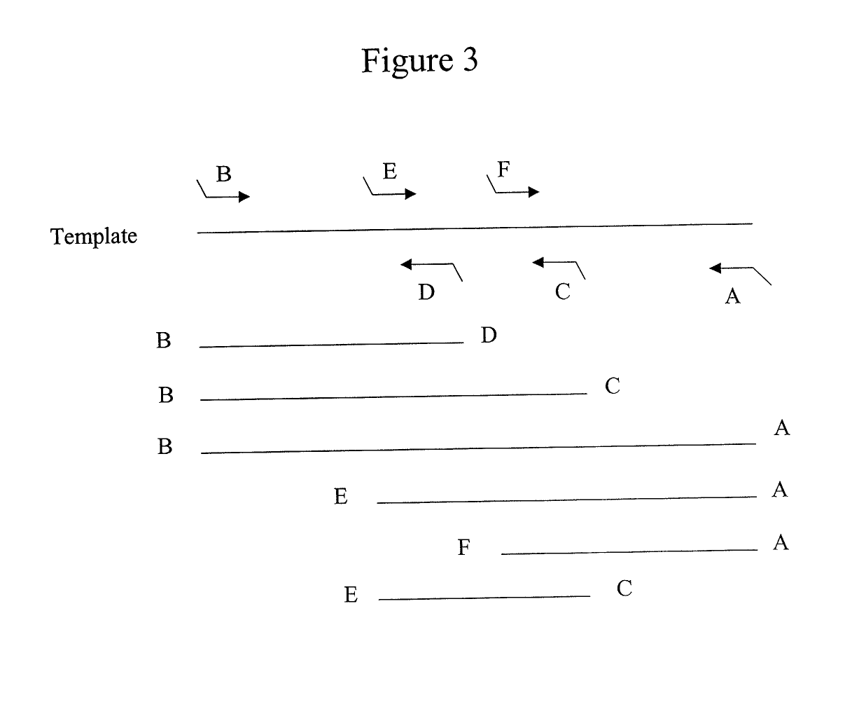

In yet another aspect of the invention the pool of 3′ blocked oligonucleotides can be prepared by copying the polynucleotide(s) of interest from end primers into a pool of variable length oligonucleotides using ddNTP to terminate copy growth. Such copying can be done in an enzyme catalyzed oligonucleotide polymerization synthesis from at least one primer which can hybridize to an end of the polynucleotide(s) of interest. The average size of the oligonucleotides produced during polymerization can be moderated by the dNTP/ddNTP ratio or by regulating the time of the extension reaction prior to increasing the ddNTP concentration. More particularly, this pool of 3′ blocked oligonucleotides of staggered length can be prepared according to the following detailed procedures and with reference to FIG. 3.

### Protocol 1

1. Denature DNA encoding the gene of interest and anneal a set of random or specific primers to the DNA.

2. Initiate DNA synthesis by adding DNA polymerase and dNTP's.

3. Allow DNA synthesis to proceed for the desired period of time, then add a large concentration of dideoxy-NTPs to the reaction. The ddNTPs will be incorporated into the growing DNA chains, thereby terminating their synthesis. The average length of the new DNA molecules will be determined by the period of time in which the reaction proceeds prior to addition of ddNTPs.

4. The DNA is denatured and the ddNTP-terminated molecules are purified away from the full-length molecules.

### Protocol 2

1. Denature DNA encoding the gene of interest and anneal a set of random or specific primers to the DNA.

2. Initiate DNA synthesis by adding DNA polymerase and a mixture of dNTP's and ddNTPs.

3. Allow DNA synthesis to proceed. The ddNTPs will be incorporated into the growing DNA chains, thereby terminating their synthesis. The average length of the new DNA molecules will be determined by the ratio of dNTPs to ddNTPs in the reaction mix.

4. The DNA is denatured and the ddNTP-terminated molecules are purified away from the full-length molecules.

The pool of 3′ blocked oligonucleotides comprises overlapping oligonucleotides along the full length of the polynucleotide(s) of interest. Polymorphic copies of the polynucleotide(s) of interest can be synthesized from the pool of 3′ blocked oligonucleotides by enzyme-catalyzed, polymerization in the absence of templates comprising the original polynucleotide(s) of interest. Such synthesis will require multiple cycles or primer extension, denaturing, reannealing to switch templates and further primer extension. The synthesis can be moderated in a variety of ways known to those skilled in the art. A preferred means of moderating synthesis is replacing the divalent cation magnesium with manganese. In other preferred aspects of this invention, the synthesis of oligonucleotides is carried out under conditions inducing mutations in the oligonucleotide as compared to the template.

The recombined, and preferably mutagenized, polynucleotide of this invention can be produced as a single-stranded molecule or as a double-stranded molecule depending on the intended application. For instance, it is often useful to screen and/or select recombined polynucleotides for a trait of interest by assessing expression in a cell or organism. Thus, a further aspect of this invention comprises introducing a produced recombined polynucleotide into a host cell, e.g. a cell having homologous DNA. Methods for using the recombined polynucleotides of this invention in whole cells and organisms are disclosed in U.S. Pat. No. 5,965,408 and International Application Publications Number WO 98/31837 and WO 00/04190, the entirety of all of which is incorporated herein by reference.

Other applications for the recombined polynucleotides of this invention, e.g. to develop cells and organisms with desired phenotypes are well known to those skilled in the art and can be found in International Application Publications Number: WO 98/42832; WO 98/57128; WO 00/09727; WO 00/09682; WO 00/12680, the entirety of all of which is incorporated herein by reference.

### EXAMPLE 1

This example serves to illustrate one embodiment of the method of this invention applied in the field of protein engineering to impart desired traits on selected proteins. Bacilllus thuringiensis (Bt) toxin proteins are important insect control agents that are frequently used in transgenic plants to provide insect pest resistance. However, insect populations often develop resistance to Bt toxins over time rendering the Bt toxins strategy ineffective To counteract the emergence of insect resistance to the toxins, new Bt toxins effective against resistant insects are needed. Such new Bt toxins with enhanced potency are obtained through the application of the present invention. A family of related Bt toxin genes are recombined in vitro according to the method illustrated in FIG. 1. The resulting recombinant Bt toxin genes are cloned in a suitable bacterial expression vector and then used to transformed an Escherichia coli (E. coli) host. Clones of E. coli harboring the recombinant Bt toxin genes are screened for enhanced insecticidal activity towards Bt toxin resistant insects. Clones that exhibit enhanced insecticidal activity are pooled and subjected to additional rounds of in vitro recombination and selection. In effect, the Bt toxin genes are subjected to directed evolution driven by the combined effects of in vitro recombination and selection. When a recombinant Bt toxin gene with enhanced insecticidal activity is identified, the recombinant gene goes go through several rounds of directed evolution to remove unnecessary mutations.

### EXAMPLE 2

This example illustrates the method of this invention where restrcition endonucleases are used to generate fragments which are useful for template switching.

The sequence of the ampicillin resistant gene TEM-1 was analyzed to identify tetra nucleotide recognition sites for restriction endonucleases which generate a 5′ overhang. See FIG. 2 for a restriction enzyme map for the TEM-1 gene. A set of five digests were prepared with the endonucleases identified in Table 2

TABLE 2

| Digest No. | Restriction Endonuclease |
|---|---|
| 1 | HinPII; Sau3AI |
| 2 | HpaII |
| 3 | MseI; AciI |
| 4 | HpaII; Sau3AI |
| 5 | HinPII |

The digests were pooled and the small and large cleavage products, e.g. fragments of less than 50 and greater than about 300 base pairs, were removed by gel purification. The saved cleaved fragments were blocked at the recessive 3′end by reacting with Klenow Fragment (3′→5′ exo^{—}) polymerase (obtained from New England BioLabs) and a ddNTP mixture (obtained from Amersham Pharmacia Biotech).

The end blocked fragments were mixed with dNTPs and a polymerase which lacks 3′→5′ exonuclease activity (AmpliTag Gold with GeneAmp 10× PCR buffer from Applied Biosystems) to provide a mixture suitable for template switching by PCR. With reference to FIG. 2 six PCR primers (primers A through F) were designed to allow production of six amplicons AB, AE, AF, BC, BD and CE. The number of cycles needed for PCR amplification is extended due to the number of cycles used for template switching to build amplicon length. For instance, detectable quantities of the shorter amplicons CE, BC, BD and AF are readily produced in about 50 to 90 PCR cycles, while about 90 to 120 PCR cycles are needed to produce detectable quantities of the longer amplicons AE and AB. Due to the long length of the original template it is more difficult to produce definitive quantities of the full length amplicon AB.

A full length gene was generated by PCR using annealed, overlapping amplicons BC and EA as the template. The PCR primers were designed to incorporate NcoI and SacI restriction sites to facilitate cloning. The SacI restriction site was incorporated at the 5′ end of the coding strand. The resulting PCR fragment was cloned into a pBR322 based vector backbone lacking the wild type TEM-1 encoding sequence. The vector was prepared by PCR amplification of pBR322 outwards from the 5′ and 3′ ends of the TEM-1 encoding region using synthetic oligonucleotides that contained overhangs containing NcoI and SacI restriction sites.

Clones resulting from the cloning process were selected on ampicillin plates. Ten of the resulting colonies were selected and their DNA analyzed by Nco1 and SacI digestion to detect the presence of cloned template switching fragment. Eight of the ten selected clones were positive for the presence of the Nco1/Sac1 TEM-1 fragment.

### EXAMPLE 3

This example illustrates the use of template switching method for incorporation of mutations in a nucleotide sequence of interest.

The TEM-1 gene conferring ampicillin resistance was digested with SficI to provide two restriction fragments of approximately equal size. A synthetic 3′ end blocked oligonucleotide was prepared which spans the SfcI restriction site of the TEM-1 gene. The oligonucleotide was designed such that incorporation of the oligonucleotide into the TEM-1 gene in place of the native sequence of the corresponding gene region would result in the generation of a HindIII restriction site in the TEM-1 gene that is not naturally present. Translation of the resulting mutated coding sequence will yield a conserved amino acid change in the TEM-1 protein.

The TEM-1 restriction fragments and the synthetic oligonucleotide were mixed in PCR buffer with TAQ polymerase. PCR was conducted and the resulting DNA product analyzed by gel electrophoresis. The analysis revealed a PCR product of the correct size for the TEM-1 gene. Restriction digestion of the PCR product with HindIII resulted in two bands of the expected size, in comparison to similar digestion of the wild-type gene which did not have a HindIII site and yielded a full length band of approximately 1 kb. This confirms that the mutation resulting in a HindIII site was incorporated into the TEM-1 gene fragment by template switching.

While the invention is described with reference to the description of preferred embodiments and illustrative example, it is not intended to be limited by the bounds of the disclosure. It is understood that a person skilled in the art will be able to produce modifications within the scope and spirit of the described invention.

What is claimed is:

1. A method for recombining sequence in a polynucleotide of interest comprising the step of generating a recombinant polynucleotide by PCR amplification using as a template a pool of oligonucleotides having a chain terminating agent at the 3′ end, wherein said recombinant polynucleotide has at least one nucleotide which is different from the nucleotide at the same position in the polynucleotide of interest.

2. A method according to claim 1 wherein said pool of oligonucleotides is produced by cutting full length polynucleotide of interest with a restriction endonuclease into fragments and adding ddNTP at the 3′ end of each fragment.

3. A method according to claim 2 wherein said restriction endonuclease provides sticky ended fragments.

4 A method according to claim 2 wherein at least two restriction endonucleases are used to provide distinct populations of said pool.

5 A method according to claim 4 wherein polymerase and a primer for one end of the template is added to said pool of oligonucleotides to form a template synthesizing mixture and subjecting said template synthesizing mixture to PCR thermal cycles to produce a pool of polymorphic, single-stranded, full length, synthesized templates.

6. A method according to claim 5 wherein said synthesized templates are amplified to build a population of double-stranded, synthesized polynucleotide having recombined sequence compared to the polynucleotide of interest.

7. A method according to claim 1 wherein said chain terminating agent is a ddNTP.

8. A method according to claim 7 wherein said pool of oligonucleotides are prepared by enzyme-catalyzed, oligonucleotide polymerization synthesis from primers in the presence of a template of said polynucleotide of interest, which template is selected from the group consisting of a single polynucleotide or a plurality of polynucleotides having different nucleotide sequences.

9. A method according to claim 8 wherein said pool of oligonucleotides is prepared by conducting enzyme-catalyzed oligonucleotide polymerization synthesis from primers in the presence of said template of said polynucleotide of interest and a mixture of dNTPs and at least one ddNTP to form said pool of oligonucleotides having a ddNTP at the 3′ end.

10. A method according to claim 9 wherein said at least one ddNTP comprises a mixture of a majority of one ddNTP and a minority of other ddNTPs.

11. A method according to claim 9 wherein said PCR amplification is carried out using 5′ end primers for said template.

12. A method according to claim 9 wherein said oligonucleotide polymerization synthesis is conducted from primers which are of random sequence or defined sequence.

13. A method according to claim 12 wherein said random sequence or defined sequence primers are blocked at the 5′ end thereof.

14. A method according to claim 13 wherein a 5′ specific exonuclease is added to said pool prior to PCR amplification to digest the original template.

15. A method according to claim 12 wherein said random sequence or defined sequence primers are oligonucleotides having a length in the range of 4 to 50 bases.

16. A method according to claim 9 producing a recombined polynucleotides which are mutagenized.

17. A method according to claim 9 producing a recombined polynucleotide as a double-stranded recombined mutagenized polynucleotide.

18. A method according to claim 9 further comprising introducing a produced recombined polynucleotide into a host cell having homologous DNA.

19. A method according to claim 8 wherein said pool of oligonucleotides is prepared in a moderated, enzyme-catalyzed, oligonucleotide polymerization synthesis from primers in the presence of said template of said polynucleotide of interest and a mixture of dNTPs, wherein said polymerization is quenched at a selected time by adding ddNTPs to a polymerization synthesis mixture to form a pool of oligonucleotides having a ddNTP at the 3′ end.

20. A method according to claim 19 wherein said enzyme-catalyzed, oligonucleotide polymerization synthesis is moderated by using manganese as a divalent cation during said synthesis.

21. A method according to claim 19 wherein said PCR amplification is carried out using 5′ end primers for said template.

22. A method according to claim 19 wherein said oligonucleotide polymerization synthesis is conducted from primers which are of random sequence or defined sequence.

23. A method according to claim 22 wherein said random sequence or defined sequence primers are blocked at the 5′ end thereof.

24. A method according to claim 23 wherein a 5′ specific exonuclease is added to said pool prior to PCR amplification to digest the original template.

25. A method according to claim 22 wherein said random sequence or defined sequence primers are oligonucleotides having a length in the range of 4 to 20 bases.

26. A method according to claim 19 producing a recombined polynucleotides which is mutagenized.

27. A method according to claim 19 producing a recombined polynucleotide as a double-stranded recombined mutagenized polynucleotide.

28. A method according to claim 19 further comprising introducing a produced recombined polynucleotide into a host cell having homologous DNA.
